SEQUENCE LISTING (15 sequences)

SEQ ID NO: 1 - DNA, 2274 bp, Arabidopsis thaliana
  CDS (1)..(2124)
atggcaagtgttcaaaaagatccgattcttcaagtagagacaacttgtggatctcttttattcgaactccagattatttgggatgaagttggagaaacagaaactgatagagatcaaatgttgcttgagcttgaacgtgaatgtttggaagtttatagaagaaaagttgatcaagctaatcgatgtagagctcagcttagacaagcaatagctgatgctgaagcacaattagctgcaatttgttctgctatgggagagcgtcctgtgcatattagacagtctgatcaaagcgttgggagcttgaagcaagagcttgggaggattcttcctgaactggaagagatgcaaaagaggaaagttgaaagaagaaaccagttcattgttgtcatggagcaaatagatagtattactaatgatattaaaggtcaaggagaacttgttcattccgaacctcttatcgatgagactaacttgtctatgagaaagcttgaagagttacactgccagctgcaagtacttcagaaggagaagattgatcgtgtggagacgattcggaagcacctgtgtactctgtattcccattgttcagtacttggaatggattttaatgaggttgttggccaagtgaatcccacattgagtgatccagaaggaccaagaagtttaagtgatcatacaattgagaaattgggggctgcagttcagaagctgatggaggtcaagatacagagaatgcagaggcttcaagatctagcaacaaccatgttggagctctggaatttgatggatacacccatagaagagcagcaagagtatcagcatattacctgcaatatagctgcatcagaacatgaaataactgaagccaatagtctctctgaagacttcattaaatacgttgaggcagaagttgtccgattggacgaggtaaaagcaagcaaaatgaaagaacttgtgttgaagaaaaggtcagaactagaggagatatgtaggaaaacccacttgttgccagtgtcagacagtgctatagatcagactatcgtggcgatagaatctggtattgtggatgctacaatggtcctggagcatcttgagcaacatatatctaagatcaaagaggaagcactaagccgaaaagagatacttgaaagggttgagaaatggttatctgcctgcgatgaagaaagttggcttgaagaatacaacagggatgataaccgatacaatgctgggcgtggagctcatcttactctcaagcgcgctgaaaaagctcgtaatcttgttaccaagctcccaggtatggttgaagcactggcctcgaagacaatagtttgggaacaagaaaacgggattgaatttctctatgatggtatccgtctcctttctatgcttgaggagtataacatactcaggcaggaaagagaagaagaacaccgtcggcaacgggatcagaaaaagcttcaaggacagctcatagcagaacaagaggcactttacgggtccaaacccagcccatctaaaccccttggtggtaaaaaggctccaagaatgtcgaccggtggtgcatctaacagaagactttccttaggagcagcaatgcatcagactcctaagcctaacaagaaagcagatcaccgacataatgatggagctttatcaaatggaagaagagggcttgacattgcaggactaccctcaaggaaacaatcaatgaatccttctgaaatgttgcaatccccattagttcgcaaaccgttttcccccatatcgaccacggtggttgcatcaaaggccaacatagcaacaacaacaacacaacaacttccgaaaaacaacgcagtgaatgagatctcatcatttgcaactcctatcaagaacaacaacattctcaggaacttggaagaagagaagatgatgactatgatgatgcaaacaccgaaaaatgttgctgcaatgattccaatcccgtcgacaccagcaacagtctctgtcccaatgcacaccgcaccaacaccattcaccaacaatgcaagattgatgtctgaaaagcctgaagttgttgagtactcgtttgaggaacgaagactcgccttcatgctacagtctgaatgtcgtttggtttgatcatcaatccgttggattcaacttaagttcttttatttgtaaaactcaaattgaatgtggtttggtttgtaaaaggtcaattatatgattggcttttttttttttttttgagtaatcatttttacacgtatggtttattctgagactgac

SEQ ID NO: 2 - protein, 707 aa, Arabidopsis thaliana
MASVQKDPILQVETTCGSLLFELQIIWDEVGETETDRDQMLLELERECLEVYRRKVDQANRCRAQLRQAIADAEAQLAAICSAMGERPVHIRQSDQSVGSLKQELGRILPELEEMQKRKVERRNQFIVVMEQIDSITNDIKGQGELVHSEPLIDETNLSMRKLEELHCQLQVLQKEKIDRVETIRKHLCTLYSHCSVLGMDFNEVVGQVNPTLSDPEGPRSLSDHTIEKLGAAVQKLMEVKIQRMQRLQDLATTMLELWNLMDTPIEEQQEYQHITCNIAASEHEITEANSLSEDFIKYVEAEVVRLDEVKASKMKELVLKKRSELEEICRKTHLLPVSDSAIDQTIVAIESGIVDATMVLEHLEQHISKIKEEALSRKEILERVEKWLSACDEESWLEEYNRDDNRYNAGRGAHLTLKRAEKARNLVTKLPGMVEALASKTIVWEQENGIEFLYDGIRLLSMLEEYNILRQEREEEHRRQRDQKKLQGQLIAEQEALYGSKPSPSKPLGGKKAPRMSTGGASNRRLSLGAAMHQTPKPNKKADHRHNDGALSNGRRGLDIAGLPSRKQSMNPSEMLQSPLVRKPFSPISTTVVASKANIATTTTQQLPKNNAVNEISSFATPIKNNNILRNLEEEKMMTMMMQTPKNVAAMIPIPSTPATVSVPMHTAPTPFTNNARLMSEKPEVVEYSFEERRLAFMLQSECRLV

SEQ ID NO: 3 - DNA, 20 bp, Artificial
  primer GUS
tccagactgaatgcccacag

SEQ ID NO: 4 - DNA, 22 bp, Artificial
  Primer DYCR
gcagttcagaagctgatggagg

SEQ ID NO: 5 - DNA, 23 bp, Artificial
  Primer DYC5RC
cctgcctgagtatgttatactcc

SEQ ID NO: 6 - DNA, 23 bp, Artificial
  Primer DYC6
ggagtataacatactcaggcagg

SEQ ID NO: 7 - DNA, 25 bp, Artificial
  Primer DYC9RC
gatgatcaaaccaaacgacattcag

SEQ ID NO: 8 - DNA, 38 bp, Artificial
  Primer Gw5pdyc
aaaaagcaggcttcacactcttccctacacaaaaccgc

SEQ ID NO: 9 - DNA, 40 bp, Artificial
  Primer Gw3pdyc
agaaagctgggtgttcgaaatgcttaagcctgtaacaggg

SEQ ID NO: 10 - DNA, 41 bp, Artificial
  Primer Gw5dycB
aaaaagcaggcttcaccatggcaagtgttcaaaaagatccg

SEQ ID NO: 11 - DNA, 37 bp, Artificial
  Primer Gw3dycK
agaaagctgggtgtcaaaccaaacgacattcagactg

SEQ ID NO: 12 - DNA, 34 bp, Artificial
  Primer Gw3dycL
agaaagctgggtgaaccaaacgacattcagactg

SEQ ID NO: 13 - DNA, 449 bp, Arabidopsis thaliana
cccaaaaccttttacttcttcgtaacggctctatttctcttctctcttctctccacacacagaaatatctcgatctaggtttctaaaattggattctctatctcaaacgctaatcgaagagttttctctgaatcgttttcttaaatctaatctcgcgaagtttcagtgacagattcaatcaaacttcaaaatcatcatcagttctggaaaatttacgagaaatctcccgtttcaggcaagttttctctcaatttgttgcttcgcaatgtcaaaatcaatcatataggctttcaatcaattgcttcgaatttagcttctaattttgagaatgacatagttagggtttcgttttccctgttacaggcttaagcatttcgaaatggcaagtgttcaaaaagatccgattcttcaagtagagacaacttgtggatctcttttattcgaactcc

SEQ ID NO: 14 - DNA, 36 bp, Artificial
  Primer MAP_RNAiGW5
aaaaagcaggcttccccaaaaccttttacttcttcg

SEQ ID NO: 15 - DNA, 36 bp, Artificial
  Primer MAP_RNAiGW3
agaaagctgggtgggagttcgaataaaagagatcca

(19) United States
(12) Patent Application Publication
(10) Pub. No.: US 2011/0030095 A1
(43) Pub. Date: Feb. 3, 2011

(54) METHOD FOR INCREASING THE RESISTANCE OF A PLANT TO ENDOPARASITIC NEMATODES

(76) Inventors: Bruno Favery, Antibes (FR); Pierre Abad, Cagnes-Sur-Mer (FR); Marie-Cécile Caillaud, Nice (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/599,879

(22) PCT Filed: May 15, 2008
(86) PCT No.: PCT/IB2008/002459

§ 371 Date: Oct. 18, 2010

(30) Foreign Application Priority Data
May 15, 2007 (EP) 07290610

(51) Int. Cl. A01H 1/00; C12N 15/82; A01H 5/00

(52) U.S. Cl. 800/279; 435/320.1; 800/298

(57) ABSTRACT

The invention relates to a method for increasing the resistance of a plant to an endoparasitic nematode by inhibiting in said plant a microtubule associated protein of the MAP65 protein family.

The present invention relates to means for enhancing the resistance of plants to nematodes.

Plant endoparasitic nematodes are small microscopic roundworms which live in the soil and infect plant roots. They can affect a broad range of species, including vegetables, cereals, fruits, flowers, and woody plants, causing important pre- and post-harvest losses. Plant endoparasitic nematodes include for instance Hirchmaniella spp, Pratylenchus spp, Radopholus spp, Ditylenchus spp, Anguina spp, Aphelenchoides spp, Bursaphelenchus spp, Rhadinaphelenchus spp, Heterodera spp, Rotylenchus spp, Tylenchulus spp, Nacobbus and Globodera spp. Among the must-damaging nematodes, one can cite the root-knot nematodes Meloidogyne spp. These nematodes establish and maintain permanent multinucleate plant feeding cells. These giant cells are essential for this obligate biotrophic pathogen growth and reproduction. After root penetration, nematode larva intercellularly migrates to induce the dedifferentiation of five to seven vascular root cells. Selected cells tremendously enlarge and become multinucleate through synchronous nuclear divisions without complete cytokinesis (Jones and Payne, 1978). Hypertrophied mature giant cell contains more than a hundred polyploid nuclei, which have also undergone extensive endoreduplication (Wiggers et al., 1990) and presents dense granular cytoplasm with numerous organelles (Jones, 1981). Surrounding giant cells, parenchyma cells hyperplasia and hypertrophy leads to typical root gall formation, the most visible infection symptom. It is not yet understood how these nematodes cause such alterations, but it is suspected that secreted parasitism proteins may have direct effects on host cells (Davis et al., 2004). Nematode feeding site ontogenesis complexity is reflected by extensive gene expression modification in infected root cells (Gheysen and Fenoll, 2002; Jammes et al., 2005). Genes involved in diverse processes such as cell cycle activation (de Almeida-Engler et al., 1999), cell wall modification (Goellner et al., 2001), hormone and defense response (Lohar et al., 2004; Jammes et al., 2005) have been identified. However unique example of gene function essential for giant cell formation has been demonstrated by knock out of the rpe gene that encodes a key enzyme in the pentose phosphate pathway (Favery et al., 1998). Giant cell cytoskeleton rearrangements have also been described to be a plant key component in compatible plant-nematode interactions (de Almeida-Engler et al., 2004; Favery et al., 2004).

Plant microtubule associated proteins MAP65 were first purified from tobacco (Nicotiana tabacum) and carrot (Daucus carota) microtubule (MT) preparation as 65-kDa proteins (Jiang and Sonobe, 1993; Chan et al., 1996). In Arabidopsis, nine genes encoding MAP65 have been identified (Hussey et al., 2002). Biochemical experiments demonstrated that MAP65s bind and bundle microtubules (MTs) in vitro (Jiang and Sonobe, 1993; Chan et al., 1996; Wicker-Planquart et al., 2004). The ability to bind MTs is dependent on a conserved motif located at the protein C-terminal half (Smertenko et al., 2004). Plant MAP65s share a large conserved domain with the yeast Anaphase spindle elongation factor 1 (Ase1p) and the human Protein Regulation Cytokinesis 1 (PRC1). Ase1p and PRC1 are involved in central spindle formation and cytokinesis (Pellman et al., 1995; Mollinari et al., 2002; Schuyler et al., 2003). Different plant MAP65s may have distinct activities and functions in relation to the different MTs arrays. Transitory expression experiments in tobacco cells revealed that several members localized to the phragmoplast, but also to other MT-based structures such as the cortical MTs, preprophase band (PPB) and mitotic spindles (Van Damme et al., 2004).

AtMAP65-3/PLEIADE, an AtMAP65 member, has been isolated by a genetic screen for root morphogenesis mutants (Müller et al., 2002). AtMAP65-3 binds to the MTs during cell division. The atmap65-3/ple mutants display cytokinesis defects in the root meristem, presumably due to compromised phragmoplast organization (Müller et al., 2004). The amino acid sequence of the AtMAP65-3 protein is available under accession number NP_{—}199973 (or gi:15242132) in the GENBANK database. It is reproduced herein as SEQ ID NO: 2. The corresponding nucleotide sequence is also available in the GENBANK database under accession number NM_{—}124539 (gi:30696073). It is reproduced herein as SEQ ID NO: 1. AtMAP65-3 comprises a MAP65_ASE1 domain (accession number pfam03999 in the CDD database, Marchler-Bauer and Bryant, 2004, or accession number IPR007145 in the InterPro database, Mulder et al., 2007) corresponding to the amino acids 36-625 of AtMAP65-3, and also comprises an AAA (ATPase) sub-domain (accession number SM00382 in the SMART database (Schultz et al., 1998 and Letunic et al., 2006) corresponding to the amino acids 6-449 of AtMAP65-3.

The Inventors have investigated the molecular mechanisms underlying giant cell formation. Using a promoter trap strategy in Arabidopsis, they identified AtMAP65-3 gene to be expressed at early stages of nematode feeding site formation. Promoter-GFP fusion analysis showed that AtMAP65-3 was expressed in all tissues enriched in dividing cells. Dwarf map65-3 mutants microscopical analysis revealed polynucleate and hypertrophied plant cells. In AtMAP65-3 absence, giant cells were induced but failed to fully differentiate and finally decayed. These giant cell defects lead to improper maturation of the infecting nematodes that depend on the nutrient supply from fully developed giant cell. Thus, the Inventors have shown that AtMAP65-3 is an essential host susceptibility gene, playing a key role in plant-nematode interaction by its requirement for giant cell development.

The present invention thus provides a method for increasing the resistance of a plant to an endoparasitic nematode, wherein said method comprises the inhibition in said plant of a microtubule associated protein of the MAP65 family, hereinafter designated as MAP65-3 protein, said MAP65-3 protein having at least 50%, preferably at least 55% and by order of increasing preference, at least 58%, 60%, 65%, 70%, 75%, 80%, 85%, 90%, 95%, 98% or 99% sequence identity, or at least 65%, preferably 70% and by order of increasing preference, at least 71%, 75%, 80%, 85%, 90%, 95%, 98% or 99% sequence similarity with the AtMAP65-3 protein (SEQ ID NO: 2).

According to a preferred embodiment, said MAP65-3 protein comprises a MAP65_ASE1 domain having at least 55%, and by order of increasing preference, at least 60%, 63%, 65%, 70%, 75%, 80%, 85%, 90%, 95%, 98% or 99% sequence identity, or at least 70%, and by order of increasing preference, at least 75%, 76%, 77%, 80%, 85%, 90%, 95%, 98% or 99% sequence similarity with the MAP65_ASE1 domain of the AtMAP65-3 protein as defined above.

According to a further preferred embodiment, said MAP65-3 protein comprises an AAA sub-domain having at least 55%, and by order of increasing preference, at least 60%, 65%, 67%, 70%, 75%, 80%, 85%, 90%, 95%, 98% or 99% sequence identity, or at least 75%, and by order of increasing preference, at least 80%, 85%, 90%, 95%, 98% or 99% sequence similarity with the AAA sub-domain of the AtMAP65-3 protein as defined above.

Unless otherwise specified, the protein sequence identity and similarity values provided herein are calculated using the BLASTP program under default parameters, on a comparison window including the whole sequence of the proteins or of the domain to be compared. Similarity calculations are performed using the scoring matrix BLOSUM62. By way of example of MAP65-3 proteins, one can cite the putative microtubule-associated protein of Solanum demissum (amino acid sequence available under accession number AAT40494 (gi:11320514) in the GENBANK database), the microtubule-associated protein-like of Oryza sativa (japonica cultivar group) (amino acid sequence available under accession number NP_{—}001043901 (gi:115439243) in the GENBANK database), or the protein of Vitis vinifera (amino acid sequence is available under accession number CAO49743 (gi:157355886) in the GENBANK database).

If necessary, a MAP65-3 protein can further be identified by determining its localization in planta (MAP65-3 locates to all microtubule arrays in dividing cells only such as the midline of the anaphase spindle and the cytokinetic phragmoplast), e.g. by the methods described in Müller et al., 2004, Van Damme et al., 2004 and/or Caillaud et al., 2008. A MAP65-3 protein can also be identified by showing that said protein enables the complementation of the Arabidopsis thaliana map65-3 mutant phenotype, e.g., by expressing said protein in the dyc283 mutant (see Example 1 below).

The inhibition of a MAP65-3 protein can be obtained either by abolishing, blocking or decreasing its function (i.e. binding to the MTs during cell division), or advantageously, by preventing or down-regulating the expression of its gene.

By way of example, inhibition of said MAP65-3 protein can be obtained by mutagenesis of the corresponding gene or of its promoter, and selection of the mutants having partially or totally lost the MAP65-3 protein activity. For instance, a mutation within the coding sequence can induce, depending on the nature of the mutation, the expression of an inactive protein, or of a protein with impaired activity; in the same way, a mutation within the promoter sequence can induce a lack of expression of said MAP65-3 protein, or decrease thereof.

Mutagenesis can be performed for instance by targeted deletion of the MAP65-3 coding sequence or promoter, or of a portion thereof, or by targeted insertion of an exogenous sequence within said coding sequence or said promoter. It can also be performed by random chemical or physical mutagenesis, followed by screening of the mutants within the MAP65-3 gene. Methods for high throughput mutagenesis and screening are available in the art. By way of example, one can mention TILLING (Targeting Induced Local Lesions IN Genomes, described by McCallum et al., 2000).

Advantageously, the inhibition of said MAP65-3 protein is obtained by silencing of the corresponding gene. Methods for gene silencing in plants are known in themselves in the art. For instance, one can mention by antisense inhibition or co-suppression, as described by way of example in U.S. Pat. Nos. 5,190,065 and 5,283,323. It is also possible to use ribozymes targeting the mRNA of said MAP65-3 protein.

Preferred methods are those wherein post transcriptional gene silencing is induced by means of RNA interference (RNAi) targeting the MAP65-3 gene to be silenced. Various methods and DNA constructs for delivery of RNAi are available in the art (for review, cf. Watson et al., 2005). Typically, DNA constructs for delivering RNAi in a plant include at least a fragment of 300 by or more (generally 300-800 bp, although shorter sequences may sometime induce efficient silencing) of the cDNA of the target gene, under transcriptional control of a promoter active in said plant. Currently, the more widely used DNA constructs are those that encode hairpin RNA (hpRNA). In these constructs, the fragment of the target gene is inversely repeated, with generally a spacer region between the repeats.

The present invention provides chimeric DNA constructs for silencing a MAP65-3 gene.

A chimeric DNA construct of the invention comprises:
a promoter functional in a plant cell;
a DNA sequence of 200 to 1000 bp, preferably of 300 to 900 bp, consisting of a fragment of a cDNA encoding a MAP65-3 protein or of its complementary, or having at least 95% identity, and by order of increasing preference, at least 96%, 97%, 98%, or 99% identity with said fragment, said DNA sequence being placed under transcriptional control of said promoter.

According to a preferred embodiment of the invention, said chimeric DNA construct comprises:
a first DNA sequence of 200 to 1000 bp, preferably of 300 to 900 bp, consisting of a fragment of a cDNA encoding a MAP65-3 protein, or having at least 95% identity, and by order of increasing preference, at least 96%, 97%, 98%, or 99% identity with said fragment;
a second DNA sequence that is the complementary of said first DNA, said first and second sequences being in opposite orientations;
a spacer sequence separating said first and second sequence, such that these first and second DNA sequences are capable, when transcribed, of forming a single double-stranded RNA molecule.

The spacer can be a random fragment of DNA. However, preferably, one will use an intron which is spliceable by the target plant cell. Its size is generally 400 to 2000 nucleotides in length.

A large choice of promoters suitable for expression of heterologous genes in plants is available in the art.

They can be obtained for instance from plants, plant viruses, or bacteria such as Agrobacterium. They include constitutive promoters, i.e. promoters which are active in most tissues and cells and under most environmental conditions, tissue or cell specific promoters which are active only or mainly in certain tissues (e.g. root cells) or certain cell types (e.g. giant cells), and inducible promoters that are activated by physical or chemical stimuli, such as those resulting from nematode infection.

One can use constitutive promoters that are commonly used in plant cells are the cauliflower mosaic virus (CaMV) 35S promoter, the Nos promoter, the rubisco promoter.

However, the most suitable promoters for silencing the MAP65-3 gene in a plant in order to increase its resistance to an endoparasitic nematode are promoters that are tissue-specific, having a preferential or exclusive expression in the tissues infected by the nematodes, in particular the giant cells, and/or promoters that are inducible by nematode infection, i.e. promoters that drive an increased level of expression in cells infected by nematodes, when compared to non-infected cells. Said promoters may be naturally occurring promoter, may comprise a nematode responsive element isolated from a naturally occurring promoter, or may be a synthetic promoter.

Non-limitative examples of preferred promoters for carrying out the invention include:
the elongation specific endo-1,4-.beta.-glucanase (cell) promoter of Arabidopsis thaliana, which is described in U.S. Pat. No. 7,119,254 as being up-regulated in root-knot nematode feeding cells;
the promoter of the POX gene of A. thaliana (Vercauteren et al., 2001);
the promoter of the Trypsin inhibitor gene of A. thaliana (Vercauteren et al., 2001);
the promoter of the endomembrane protein gene of A. thaliana (Vercauteren et al., 2001);
the promoter of the DAP decarboxylase gene of A. thaliana (Vercauteren et al., 2001);
the promoter of the WRKY23 (Att0001) gene of A. thaliana (Barthels et al., 1997); or preferably
the promoter of the AtFH6 gene (Favery et al., 2004), also disclosed in WO 2005/063989 (SEQ ID NO: 1 of WO 2005/063989).

The DNA constructs of the invention generally also include a transcriptional terminator (for instance the 35S transcriptional terminator, or the nopaline synthase (Nos) transcriptional terminator).

These DNA constructs can be obtained and introduced in a host cell or organism by the well-known techniques of recombinant DNA and genetic engineering. The choice of the recombinant vector depends on the intended host and on the intended method of transformation of said host. A variety of methods for genetic transformation of plant cells or plants are available in the art for many plant species, dicotyledons or monocotyledons. By way of non-limitative examples, one can mention virus mediated transformation, transformation by microinjection, by electroporation, microprojectile mediated transformation, Agrobacterium mediated transformation, and the like.

The invention also comprises plant cells or plants genetically modified by a construct of the invention. The polynucleotide may be transiently expressed; it can also be incorporated in a stable extrachromosomal replicon, or integrated in the chromosome.

In particular the invention relates to a transgenic plant containing a transgene comprising a chimeric DNA construct of the invention. The expression of said chimeric DNA constructs, resulting in a down regulation of the MAP65-3 protein, provides to said transgenic plant an increased resistance to endoparasitic nematodes when compared with a plant devoid of said transgene.

The present invention applies to monocot- or dicotyledon plants of agronomical interest, such as wheat, maize, rice, oats, barley, rye, sorghum, triticale, sugar cane, tomato, potato, sugarbeet, rape, flax, tobacco, sunflower, cotton, peas, beans, soybean or alfalfa.

The invention also encompasses isolated organs or tissues of said plants (such as seeds, leafs, flowers, roots).

Foregoing and other objects and advantages of the invention will become more apparent from the following detailed description and accompanying drawings. It is to be understood however that this foregoing detailed description is exemplary only and is not restrictive of the invention.

### DESCRIPTION OF THE DRAWINGS

FIG. 1. DYC283 T-DNA-Tagged Arabidopsis Line Displaying GUS Activity in Galls Induced by M. incognita and during Plant Development. (A-C) GUS expression in galls induced by M. incognita. Localized GUS activity in a root gall 7 days post infection (dpi) (A). Sectioned gall shown in (A) seen by dark-field microscopy (B). GUS activity (seen as a pink precipitate) is observed in the giant cells and in the surrounding cells. Sectioned galls 21 dpi observed in dark-field microscopy. Asterisks, giant cells; N, nematode. (C). GUS activity is observed in surrounding cells and not in mature giant cells. Asterisks, giant cells; N, nematode. (D) Organization of the AtMAP65-3 gene and molecular analysis of the T-DNA insertion. Boxes indicate the exons. GUS corresponds to the coding region of the β-glucuronidase gene present on the T-DNA. The initiation and stop codons are indicated. (E-I) GUS expression during plant development. Reporter gene activity was observed in root meristem (E), lateral root meristem (F) developing leaves of a 14-day-old seedling (G), buds (H) and ovules (I). Bars in (A)=100 μm; (B) and (C)=25 μm; in (E) and (F)=50 μm; in (H) and (I)=150 μM, in (G)=1 cm.

FIG. 2. Gene Expression Patterns Observed in AtMAP65-3 Promoter:GFP:GUS Fusion Line. (A-C) GFP expression in the root, in root meristem (A), lateral root emergency (B) and lateral root meristem (C). (D-F) GFP expression in the shoot, in the shoot apical meristem (D), young leaves (E). In mature leaves GFP signal is located in meristemoids, stomata in formation and in mature stomata (F). (G-I) GFP expression during embryogenesis, in endosperm and embryo (G), in heart embryo stage (H) and in late torpedo stage (I). Bars in (A) to (D) and (G) to (I)=50 μm, in (E)=200 μm, in (F)=10 μm.

FIG. 3. AtMAP65-3 Interruption Leads to a Shoot and Root Mutant Phenotype. (A-E) dyc283 mutant shoot phenotype in plants homozygous for the T-DNA mutation (dyc283/dyc283). The dyc283 mutant showed a strong reduction of the shoot in comparison with wild type plant (A). Hypocotyl and leaf primordial (B-C) exhibited polynucleate cells and aberrant cell wall stubs (arrows). In leaf primordia, nuclei were often irregular lobed and amoeboid in form (D). Mature leaf stained with DAPI, major part of mesophyll cells was binucleate and some cells presented four nuclei (E) (arrows). (F-K) dyc283 mutant root phenotype. Longitudinal section through wild type root (F). Longitudinal section through dyc283 mutant revealed multinucleate hypertrophied cortical and epidermal cells (arrows) (G). Transversal section through mutant root stained with DAPI showed polynucleate (arrows) and hypertrophied cells (H). Vascular cylinder cells often show cell misalignments (arrows) (H). dyc283 mutant expressing H2B:YFP and stained with membrane dye FM4-64 exhibited multinucleate root cells and cell wall stubs (I). dyc283 mutant root electron myograph showed abnormal enlarged nuclei, that contained nucleolus number increase (arrows) (J), non-divided nucleus (arrow) curving around incomplete wall branched end (K). nu, nucleus; vac, vacuole; cws, cell wall stub; cw, cell wall. Bars in (A)=2 cm; in (B), (F) and (G)=50 μm; in (C) to (E), (H) and (I)=20 μm; in (J) and (K)=1 μm.

FIG. 4. Giant Cells and Root-Knot Nematode Mutant Phenotype (A-B) Visualization of nuclei in young giant cells using plant expressing H2B:YFP. In control wild-type plant, giant cell was binucleate (A). In dyc283 mutant plant, giant cells were mononucleate with swollen nuclei (B). (C-D) Cross section through gall of wild-type plant (C) and dyc283 plant (D), 7 dpi with M. incognita. Arrows showed aberrant cell wall stubs in dyc283 mutant giant cell (D). (E-F) Cross section through gall of wild type plant (E) and dyc283 mutant plant (F), 21 dpi with M. incognita. In control wild-type plant, giant cells were mature and nematode was at the fourth juvenile stage (E). In the dyc283 mutant plant, giant cells decayed and nematode stayed at the third stage (F). Asterisks, giant cells; N, nematode. Bars in (A) to (D)=20 μm; in (E) and (F)=40 μm.

FIG. 5. Pro_{AtFH6}-RNAi AtMAP65-3 T-DNA construct used for the generation of transgenic plants with the MAP65-3 gene silenced in giant cells.

### EXAMPLES

### Experimental Procedures

### Plant Materials, Growth Conditions, and Nematode Infection

The T-DNA mutagenized Arabidopsis thaliana line collection (ecotype Wassilewskija, Wis.) was generated at Institut National de la Recherche Agronomique Versailles for promoter trap and gene tagging (Bechtold et al., 1993). The lines were screened individually for GUS expression after Meloidogyne incognita infection as previously described (Favery et al., 1998). For in vitro analyses, seeds were surface sterilized and grown on MS medium containing 1% sucrose, 0.7% plant cell culture tested agar (Sigma, St. Louis, Mo., USA), and 50 μg/mL of kanamycin. Plates were inclined at an angle of 60° to allow the roots to grow along the surface. Kanamycin resistance was scored in 2-week-old seedlings. For nematode infection in vitro, 100 surface-sterilized freshly hatched M. incognita J2 were added on each 2-week-old seedling. The plates were kept at 20° C. with a 16-h photoperiod.

### Histochemical Localization of GUS Activity and Microscopic Analyses

GUS activity was assayed histochemically with 5-bromo-4-chloro-3-indolyl-β-D-glucuronic acid as described by Favery et al. (1998). Galls, root apex, and shoot apical meristems were dissected from GUS-stained plants, fixed in 1% glutaraldehyde and 4% formaldehyde in 50 mM sodium phosphate buffer, pH 7.2, dehydrated, and embedded in Technovit 7100 (Heraeus Kulzer, Wehrheim, Germany) as described by the manufacturer. Sections (4 μm) were stained with 0.05% ruthenium red or toluidine blue and mounted in DPX (BDH Laboratory Supplies, VWR International, Fontenay-sous-Bois, France). Sections were observed with a Zeiss Axioplan 2 microscope (Jena, Germany).

T-DNA Insertion Site Analysis and Isolation of Homozygous dyc283 and ebj96 Plants

The insertion site was characterized by sequencing the genomic regions flanking the inserted T-DNA as described by Samson et al. (2002). To isolate homozygous dyc283/dyc283 and ebj96/ebj96 plants, the segregation of the kanamycin marker carried by the T-DNA on progenies resulting from each of 20 plants was analyzed. Progenies of five plants segregated 100% kanamycin-resistant plants, indicating that they were homozygous for the T-DNA tagged allele. To confirm these results, PCR experiments were done with the AtMAP65-3 primers which span the T-DNA insertion site, and a third primer GUS (5′-tcc aga ctg aat gcc cac ag-3′, SEQ ID NO: 3) specific for the sequence of the T-DNA. The primers DYCR (5′-gca gtt cag aag ctg atg gag g-3′, SEQ ID NO: 4) and DYC5RC (5′-cct gcc tga gta tgt tat act cc-3′, SEQ ID NO: 5) were used for DYC283, and DYC6 (5′-gga gta taa cat act cag gca gg-3′, SEQ ID NO: 6) and DYC9RC (5′-gat gat caa acc aaa cga cat tca g-3′, SEQ ID NO: 7) for EBJ96. When genomic DNA from homozygous plants was used as a template, a single PCR product was obtained from amplifications with all three primers.

### Transgenic Plants and Crosses

For AtMAP65-3 promoter GFP:GUS fusion, a fragment of 1240 by upstream of the start codon was amplified by PCR using the primers Gw5pdyc (5′-aaa aag cag get tca cac tct tee cta cac aaa acc gc-3′, SEQ ID NO: 8) and Gw3pdyc (5′-aga aag ctg ggt gtt cga aat get taa gcc tgt aac agg g-3′, SEQ ID NO: 9). The PCR fragment was inserted into the pDON207 donor vector and then in the plant expression vectors pKGWFS7 (Karimi et al., 2002) using Gateway Technology (Invitrogen). For the subcellular localisation of MAP65-3, the Pro_{35S} HindIII/SpeI fragment of the pK7WGF2, pK7FWG2, pH7WGY2, pH7YWG2 vectors (Karimi et al., 2002) was replaced by Pro_{MAP65-3}. The coding sequence of MAP65-3 was amplified by PCR, using wild-type plant cDNAs as the template and the primers Gw5dycB (5′-aaa aag cag get tca cca tgg caa gtg ttc aaa aag atc cg-3′, SEQ ID NO: 10) and Gw3dycK (5′-aga aag ctg ggt gtc aaa cca aac gac att cag act g-3′, SEQ ID NO: 11) or Gw3dycL (5′-aga aag ctg ggt gaa cca aac gac att cag act g-3′, SEQ ID NO: 12) for GFP or YFP:AtMAP65-3 or AtMAP65-3:GFP or YFP fusion respectively. These sequences were inserted into the pDON207 donor vector and then in the Pro_{MAP65-3} plant expression vectors using Gateway Technology (Invitrogen). These constructs were sequenced by Genome Express (Grenoble, France) and transformed into Agrobacterium tumefaciens strain GV3101. Wild type WS, homozygous dyc283 or ebj96 Arabidopsis plants were transformed using the dipping method (Clough and Bent, 1998) and selected on MS medium 0.7% agar plates containing 50 μg/ml kanamycin or hygromycin. Transformed plants were transferred to soil and seeds collected. Transgenic plants expressing Pro_{MAP65-3}:AtMAP65-3:GFP or the N-terminal domain of the MT-binding domain of MAP4 fused to the GFP (Pro_{35S}:MBD:GFP) were crossed with Pro_{35S}:H2B:YFP Arabidopsis plants. Plants expressing the two constructions were obtained and homozygous progeny was used for microscopy analysis.

### Confocal Microscopy

Optical sections were obtained on fresh roots by using an inverted confocal microscope (model LSM510; Zeiss, Jena, Germany). To visualize the MT cytoskeleton and nuclei in nematode feeding site, galls of 7 and 14 dpi were excised and embedded in agar 7%. Vibroslices of 100 μm diameter—for galls 7 dpi—or 300 μm diameter—for galls 14 dpi—were obtained using a vibratome HM560V (Microm). Fresh roots and vibroslices were observed with a ×63 water immersion apochromat objective (Zeiss, Jena, Germany). YFP and GFP fluorescence were monitored in Lambda mode with a 499-550-nm beam path (488-nm excitation line). Fluorescent dye FM4-64 (Molecular probes) was used at 1 μM final concentration. GFP or YFP and FM4-64 fluorescence were monitored in Lambda mode with a 499-620-nm beam path (488-nm excitation line).

### Example 1

### The DYC283-Tagged Line Displayed GUS Activity in the Nematode Feeding Site

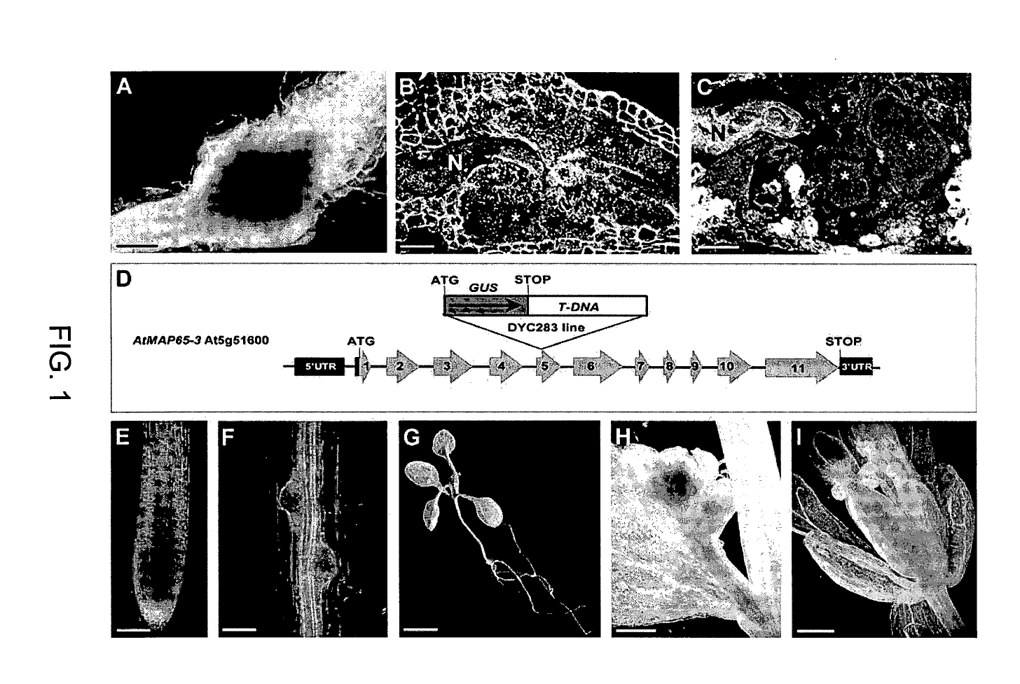

To isolate genes involved in giant cell formation induced by Meloidogyne incognita, a promoter trap strategy was developed. 20,000 T-DNA-tagged Arabidopsis lines were screened by GUS assay after root-knot nematode infection, and lines showing GUS induction in root galls (Favery et al., 2004) were identified. One of these lines, DYC283, displayed early GUS activity in galls (FIG. 1A), that was detected less than 48 h after giant cell initiation. Sections through galls 7 days post infection (dpi) clearly showed GUS staining in developing giant cells and in dividing surrounding cells (FIG. 1B). Later during the interaction, 21 dpi, GUS expression was detected only in surrounding cells and disappeared in mature giant cells (FIG. 1C). DYC283 line molecular and genetic analysis showed that this line carried a single T-DNA insert (data not shown). The insertion site was characterized by sequencing the genomic regions flanking the inserted T-DNA (FST project, Samson et al., 2002). Sequence analysis, using the Arabidopsis Resource (TAIR), showed that the T-DNA had integrated into the predicted At5g51600 gene, named AtMAP65-3. This gene has 11 exons and encodes a 707 amino acid long protein AtMAP65-3, which is a member of the MT-associated protein MAP65 family (Müller et al., 2004). In the DYC283 line, the T-DNA was inserted into the fifth exon, placing the GUS gene ATG in frame with AtMAP65-3 gene and resulting in a functional gene fusion (FIG. 1D).

### Example 2

### AtMAP65-3 is Expressed in all Tissues Enriched in Dividing Cells

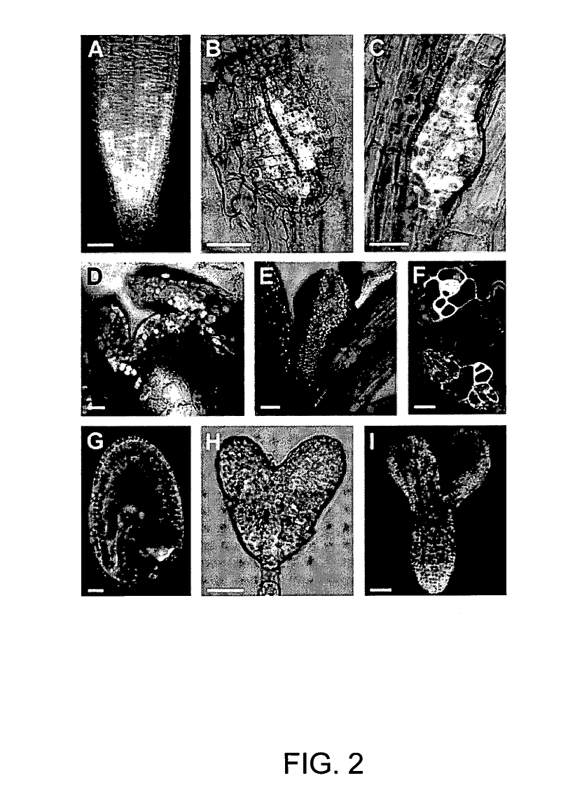

During plant development, the GUS gene was expressed in the root meristem and in the root elongation zone, in which cells divide and expand (FIG. 1E). GUS activity was also detected in the lateral root primordia (FIG. 1F). In aerial parts of the plant, GUS expression was observed in young leaves, buds and flowers (FIG. 1G-I). The AtMAP65-3 expression pattern deduced from experiments with the GUS reporter gene was confirmed using RT-Q-PCR analysis (data not shown) and AtMAP65-3 promoter (Pro_{MAP65-3}) GFP fusion. Arabidopsis plants transformed with a Pro_{MAP65-3}:GFP:GUS construct presented an expression pattern in accordance with the GUS expression observed in DYC283 line (FIG. 2A-E). GFP expression pattern analysis showed that AtMAP65-3 was expressed in all tissues enriched in dividing cells, such as shoot apical meristem (SAM) (FIG. 2D), foliar primordia and young leaves (FIG. 2E). In older leaves, expression was restricted to meristemoid and mature stomata (FIG. 2F). During embryogenesis, GFP signal was observed at early stage post fertilization, in the embryo and in the syncytial endosperm (FIG. 2G). In torpedo stage, GFP signal was uniform (FIG. 2H) whereas in late torpedo stage, GFP signal was observed through more-intense staining in the zone corresponding to root meristem (FIG. 2I).

### Example 3

### AtMAP65-3 Interruption Leads to Polynucleate and Hypertrophied Plant Cells

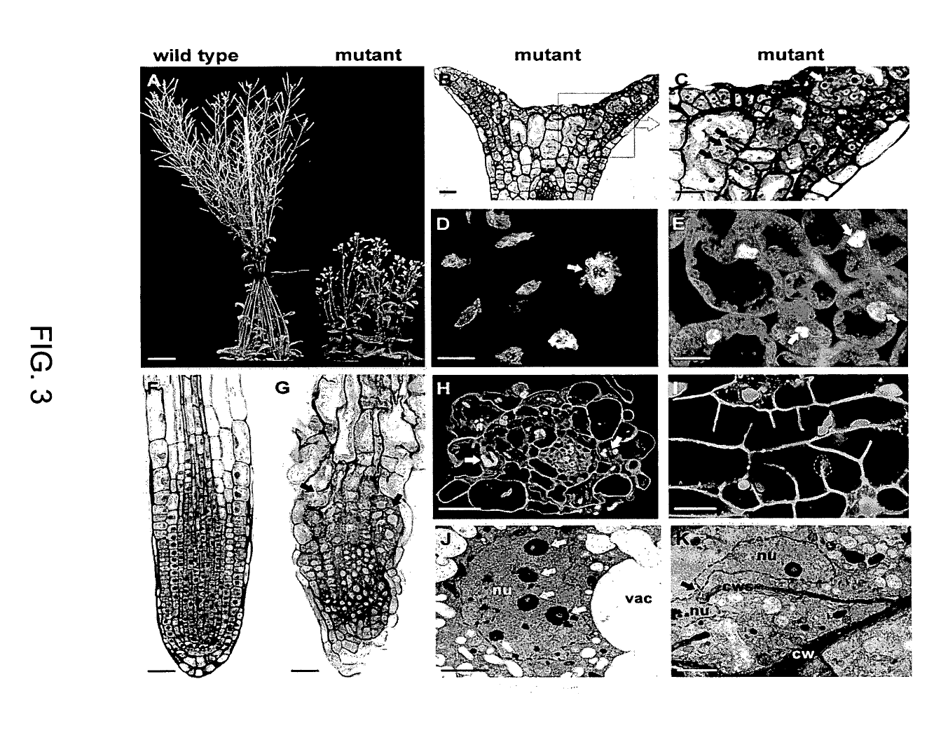

Plants homozygous for the T-DNA mutation (dyc283 mutant) showed a strong reduction of the shoot part of the plant (FIG. 3A). Despite this dwarf phenotype, no organ fusions or abnormal organ numbers have been observed. dyc283 mutant was fertile, but the number of seeds per silique was strongly reduced. Microscopical analysis revealed that the embryo sac decayed at early steps after fertilization (data not shown).

dyc283 mutant cellular organization was characterized by performing sections through different tissues. dyc283 mutant SAM presented irregular cell outer layers. Hypocotyl and leaf primordia exhibit polynucleate cells and aberrant cell wall stubs (FIG. 3B-C), absent in wild type. In young tissues, like in leaf primordial, nuclei were often irregular lobed and amoeboid in form (FIG. 3D). In mature leaves, major part of mesophyll cells was binucleate and some cells presented, at least, four nuclei (FIG. 3E). Cell wall stubs and polynucleate cells were also observed during embryogenesis in dyc283 mutant line. dyc283 mutant has also a short and swollen root system with few lateral roots (FIGS. 3F and 3G). Section through elongation zone revealed polynucleate hypertrophied cortical and epidermal cells (FIG. 3H). Vascular cylinder cells, despite being less affected by the mutation, often showed cell misalignments. Root cells were often multinucleate and exhibited cell wall stubs (FIGS. 3H and 3I). Electron microscopy performed in dyc283 mutant root confirmed presence of abnormal enlarged nuclei that contained nucleolus number increase (FIG. 3J). In addition, some dyc283 mutant cells exhibited non-divided nucleus curving around incomplete wall branched end (FIG. 3K). Organ cross sections performed in DYC283 heterozygous plants revealed no aberrant cellular phenotype confirming the recessive nature of the mutation (data not shown). Thus dyc283 mutant showed a cytokinesis defect characterized by cell wall stubs, polynucleate and hypertrophied cells in all plant organs.

### Example 4

### In Absence of AtMAP65-3, Giant Cells were Induced but Failed to Fully Differentiate

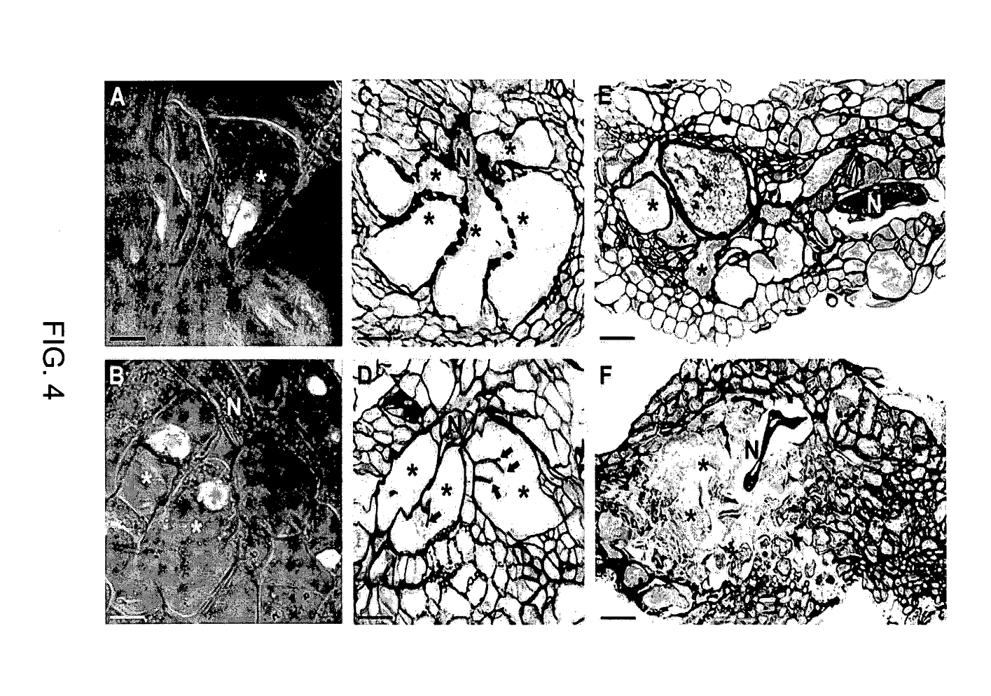

dyc283 mutant response to the nematode M. incognita was examined. The infective second stage juvenile (J2) was able to invade the dyc283 mutant root tissue by penetrating the elongation zone, migrating along the vascular cylinder and induce a gall, as well as in wild type plant. Observation of gall sections 7 dpi revealed that the nematode could initiate its feeding site composed of 5 to 7 giant cells (FIG. 4A). At this stage, giant cells in the dyc283 mutant were slightly smaller than wild type (FIG. 4B), and were characterized by unusual cell wall stubs (FIG. 4A). At the early step of giant cell initiation, unique enlarged nucleus is observed in the dyc283 mutant giant cell (FIG. 4C) when wild type giant cells were binucleate (FIG. 4D). Once the nematode feeding site was initiated, J2 stage nematodes became sedentary and developed into third stage juvenile. Later during the interaction, when nematodes develop into the fourth developmental stage in wild type, dyc283 mutant gall sections revealed that nematode development was arrested at the third stage juvenile. No fourth juvenile stage has ever been observed in dyc283 mutant. Degenerated giant cells and dead nematodes were observed 21 dpi (FIG. 4E). Mature giant cells have never been found as observed in wild type (FIG. 4F). Consequently, nor female, nor egg masses could be observed on the dyc283 mutant root surface. This incomplete root-knot nematode development indicated a defect in dyc283 mutant giant cell formation.

### Example 5

### AtMAP65-3:GFP Fusions Complement the Mutant Phenotype

Searching Arabidopsis mutant collections for loss-of-function insertion mutants, an additional map65-3 allele was identified from INRA Versailles (Samson et al., 2002). The EBJ96 line carried a single T-DNA insertion in the AtMAP65-3 eleventh exon. As expected, plants homozygous for the ebj96 mutation exhibited similar mutant phenotype to dyc283 mutant. To complement the mutant phenotype, two AtMAP65-3:GFP fusions, GFP:AtMAP65-3 and AtMAP65-3:GFP, were constructed under the Pro_{MAP65-3} control. When introduced into dyc283 or ebj96 mutants both constructs restored wild type phenotype. These results demonstrated that the map65-3 recessive mutation was responsible to the observed phenotype.

### Example 6

### Generation of Transgenic Plants with the Map65-3 Gene Silenced

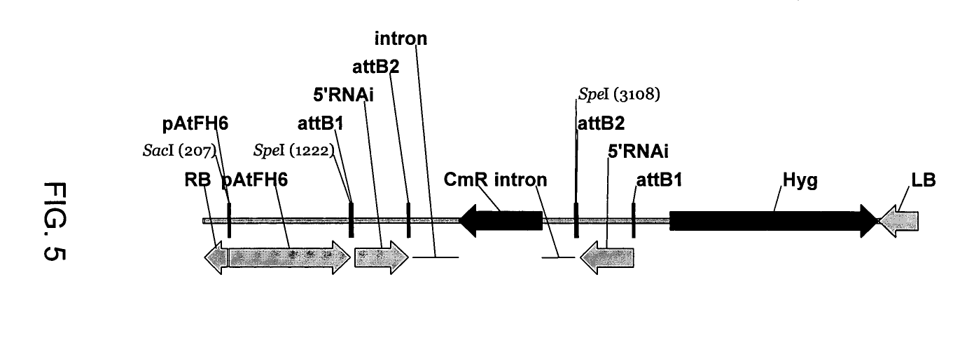

For AtMAP65-3 specific silencing in giant cells, the Pro_{35S} Sad/SpeI fragment of the pH7GWIWG2 (II) vectors (VIB Gent, Karimi et al., 2002) was replaced by the promoter of AtFH6. The 449 by 5′UTR-exonI sequence of MAP65-3 (SEQ ID NO: 13) was amplified by PCR, using wild-type plant genomic DNA as the template and the primers MAP_RNAiGW5 (5′-aaa aag cag get tcc cca aaa cct ttt act tct tcg-3′, SEQ ID NO: 14) and MAP_RNAiGW3 (5′-aga aag ctg ggt ggg agt tcg aat aaa aga gat cca-3′, SEQ ID NO: 15)—AtMAP65-3 specific sequence underlined. This sequence was inserted into the pDON207 donor vector and then in the Pro_{AtFH6}-pH7GWIWG2 (II) vector using Gateway Technology (Invitrogen). The Pro_{AtFH6}-RNAi AtMAP65-3 construct (FIG. 5) was sequenced and transformed into Agrobacterium tumefaciens strain GV3101. Pro_{MAP65-3}:GFP-GUS plants were transformed using the dipping method (Clough and Bent, 1998) and selected on MS medium 0.7% agar plates containing 50 μg/ml hygromycin. Transformed plants are transferred to soil and seeds collected. The efficiency of AtMAP65-3 silencing in roots is scored using GUS staining and GFP microscopy observation of transformed Pro_{MAP65-3}:GFP-GUS plants.

T2 plants from 19 T1 transformants obtained with the Pro_{AtFH6}-RNAi AtMAP65-3 construct showed a significant reduction of the GUS expression limited to the galls induced by the nematodes. As expected, the AtFH6 promoter being not activated in the root meristems, no reduction of the GUS activity was observed in the root meristems and no developmental phenotype was detected. The first test of infection by the root knot nematodes performed on the T2 plants coming from 10 T1 plants bearing the Pro_{MAP65-3}:GFP-GUS and Pro_{AtFH6}-RNAi AtMAP65-3 constructs allowed identifying a line with a reduction of 60% of the number of nematodes able to carry out their development cycle (2 months after infection).

### REFERENCES

Barthels et al., Plant Cell 9, 2119-2134 (1997).
Bechtold et al., C. R. Acad. Sci. Paris 316, 1194-1199 (1993).
Caillaud et al., Plant Cell 20, 423-437 (2008).
Chan et al., Plant J. 10, 251-259 (1996).
Clough and Bent, Plant J. 16, 735-43 (1998).
Davis et al., Trends Parasitol. 20, 134-141 (2004).
de Almeida-Engler et al., Cell 11, 793-807 (1999).
de Almeida-Engler et al., Plant J. 38, 12-26 (2004).
Favery et al., Embo J. 17, 6799-6811 (1998).
Favery et al., Plant Cell 16, 2529-2540 (2004).
Gheysen and Fenoll, Annu. Rev. Phytopathol. 40, 191-219 (2002).
Goellner et al., Plant Cell 13, 2241-2255 (2001).
Hussey et al., Plant Mol. Biol. 50, 915-924 (2002).
Jammes et al., Plant J. 44, 447-458 (2005).
Jiang and Sonobe, J. Cell Sci. 105, 891-901 (1993).
Jones, New York: Academic Press, pp. 225-279 (1981).
Jones and Payne, J. Nematol. 10, 70-84 (1978).
Karimi et al., Trends Plant Sci. 7, 193-195 (2002).
Letunic et al., Nucleic Acids Res 34, D257-D260 (2006).
Lohar et al., Plant J. 38, 203-214 (2004).
Marchler-Bauer and Bryant, Nucleic Acids Res. 32, 327-331 (2004).
McCallum et al., Plant Physiol., 123, 439-442 (2000).
Mollinari et al., J. Cell Biol. 157, 1175-1186 (2002).
Mulder et al., Nucleic Acids Res. 35 (Database Issue), D224-228 (2007).
Müller et al., Plant Physiol. 130, 312-324 (2002).
Müller et al., Cum Biol. 14, 412-417 (2004).
Pellman et al., J. Cell Biol. 130, 1373-1385 (1995).
Samson et al., Nucleic Acids Res. 30, 94-97 (2002).
Schultz et al., Proc. Natl. Acad. Sci. USA 95, 5857-5864 (1998).
Schuyler et al., J. Cell Biol. 160, 517-528 (2003).
Smertenko et al., Plant Cell 16, 2035-2047 (2004).
Van Damme et al., Plant J. 40, 386-398 (2004).
Vercauteren et al., Mol Plant Microbe Interact 14, 288-299 (2001).
Watson et al., FEBS Letters, 579, 5982-5987 (2005).
Wicker-Planquart et al., Plant J. 39, 126-134 (2004).
Wiggers et al., Phytopathology 80, 1391-1395 (1990).

1. A method for increasing the resistance of a plant to an endoparasitic nematode, wherein said method comprises the inhibition in said plant of a microtubule associated protein of the MAP65 family, said protein hereinafter designated as MAP65-3 protein, having at least 50% sequence identity or at least 71% sequence similarity with the AtMAP65-3 protein of SEQ ID NO: 2.

2. A method according to claim 1, wherein the inhibition of said MAP65-3 protein is obtained by expressing in said plant RNAi targeting the gene encoding said protein.

3. A chimeric DNA construct comprising:
a promoter functional in a plant cell;
a DNA sequence of 200 to 1000 bp, selected among:
a) a fragment of a cDNA encoding a MAP65-3 protein as defined in claim 1, or of the complementary of said cDNA, or
b) a fragment of DNA having at least 95% identity with said fragment a); said DNA sequence being placed under transcriptional control of said promoter.

4. A chimeric DNA construct of claim 3, comprising:
a first DNA sequence of 200 to 1000 bp, consisting of a fragment of a cDNA encoding a MAP65-3 protein as defined in claim 1, or having at least 95% identity with said fragment;
a second DNA sequence that is the complementary of said first DNA, said first and second sequences being in opposite orientations;
a spacer sequence separating said first and second sequences, such that these first and second DNA sequences are capable, when transcribed, of forming a single double-stranded RNA molecule.

5. A chimeric DNA construct of claim 3, wherein the promoter is a nematode inducible promoter.

6. A recombinant vector comprising a chimeric DNA construct of claim 3.

7. A transgenic plant containing a transgene comprising a chimeric DNA of claim 3.

8. A chimeric DNA construct of claim 4, wherein the promoter is a nematode inducible promoter.

9. A recombinant vector comprising a chimeric DNA construct of claim 4.

10. A recombinant vector comprising a chimeric DNA construct of claim 5.

11. A transgenic plant containing a transgene comprising a chimeric DNA of claim 4.

12. A transgenic plant containing a transgene comprising a chimeric DNA of claim 5.
